(19) United States
(12) Patent Application Publication
(10) Pub. No.: US 2024/0215983 A1
(43) Pub. Date: Jul. 4, 2024

(54) OPTIMIZED TRANSSEPTAL PUNCTURE LOCATION

(71) Applicant: BIOSENSE WEBSTER (ISRAEL) LTD., YOKNEAM (IL)

(72) Inventors: Assaf Govari, Haifa (IL); Andres Claudio Altmann, Haifa (IL); Eid Adawi, Tur'an (IL); Ana Kaufman, Zichron Ya'akov (IL); Jouna Khalil, Haifa (IL); Vitaly Gutkovich, Haifa (IL); Guy Haiman, Haifa (IL); Guy Wekselman, Tel Aviv (IL)

(21) Appl. No.: 18/092,977

(22) Filed: Jan. 4, 2023

(51) Int. Cl. A61B 17/12; A61B 17/00

(52) U.S. Cl. CPC A61B 17/12122; A61B 17/00234; A61B 2017/00243

(57) ABSTRACT

A system includes a display and a processor. The display is configured to present a rendering of at least part of a septum and a left atrium of a heart of a patient. The processor is configured to (a) identify in the rendering (i) the septum and (ii) a target anatomical location to be reached by a probe via the septum, (b) calculate a trajectory for the probe between the septum and the target anatomical location, including identifying over the septum an entrance location for the probe to cross the septum and reach the target anatomical location, and (c) present the entrance location to a user for penetrating the septum therein.

### FIELD OF THE DISCLOSURE

The present disclosure relates generally to treatment planning for a medical probe, and particularly to planning and/or guiding a transseptal access for an invasive medical device.

### BACKGROUND OF THE DISCLOSURE

Various methods for planning catheter approach to the left atrium (LA) to perform a catheterization-based treatment therein, such as LA appendage (LAA) occlusion or pulmonary vein (PV) electrophysiological isolation, were proposed in the patent literature. For example, U.S. Patent Application Publication 2022/0133261, which is assigned to the assignee of the current application, describes a method that includes, using a processor, identifying a septum and an LAA of a heart of a patient in an anatomical map of at least part of the heart. An entry surface over which a medical device is defined on the anatomical map, which is to be delivered via a sheath that penetrates the septum, is to engage with the LAA. A normal to the entry surface is calculated. A plurality of curves is calculated that each (i) have one end that is tangent to the normal, (ii) have a second end touching the septum, and (iii) comply with specified mechanical properties of the sheath. Multiple candidate locations on the septum are derived from the curves, for transseptal puncture with the sheath. The multiple candidate locations are presented to a user.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheterization system comprising a catheter carrying a device inside the left atrium (LA), the catheter inserted into the LA using transseptal access, in accordance with an example of the present disclosure;

FIG. 2 is a schematic, pictorial illustration of a catheter at an optimized transseptal puncture location, in accordance with an example of the present disclosure;

FIG. 3 is a flow chart that schematically describes a method of finding the optimized transseptal puncture location described in FIG. 2, in accordance with an example of the present disclosure;

FIG. 4 is a schematic, pictorial illustration of a catheter at an optimized transseptal puncture location, in accordance with another example of the present disclosure; and

FIG. 5 is a flow chart that schematically describes a method of finding the optimized transseptal puncture location described in FIG. 4, in accordance with an example of the present disclosure.

### DETAILED DESCRIPTION OF EXAMPLES

### Overview

Catheterization is an established therapy to perform treatments inside the left atrium (LA). To access the LA with a catheter, a physician typically first introduces the catheter to the right atrium (RA) via the body vasculature, and pierces the septum dividing the left and right atria with a sheath of the catheter. The physician then threads the sheath into the LA through the pierced location and delivers, via a sheath, a medical device (e.g., a catheter fitted with electrodes) to engage with LA tissue.

The physician must further carefully consider the transseptal piercing location, since its location may affect the ability to control a catheter inside the LA. Proper selection of the transseptal piercing location is important, as it substantially impacts the acquisition of several treatment milestones, such as the ease of advancing the sheath into a particular LA target location, stable contact with septum wall tissue, and maintaining catheter position at the target LA location during the invasive treatment. A combination of the above considerations, together with a given medical profile of the patient, may therefore limit a physician's options to perform a successful catheterization process in the LA.

An invasive procedure that requires particularly careful consideration of the transseptal piercing location is occlusion of the left atrial appendage (LAA). Such a procedure is used to reduce the probability of blood clots forming in the appendage, which is likely to happen in certain patients with AF. The LAA is occluded by a catheter deploying an LAA occlusion device. The occlusion procedure requires careful angular alignment of the catheter relative to an ostium of the LAA in order to have a successful outcome. However, because of limitations of the flexibility and maneuverability of the sheath and the catheter, it is not simple to successfully navigate a catheter to the LAA from a suboptimal septum piercing location.

Examples of the present disclosure that are described hereinafter provide techniques to optimize selection of a transseptal puncture location.

In the disclosed techniques, it is assumed that the puncture is made in a location over the fossa ovalis region. The fossa ovalis is a depression in the right atrium of the heart at the level of the interatrial septum. The assumption means that a location over the catheter shaft coincides with a fossa ovalis location.

In one example, the disclosed technique assumes that the ease of access to a target location inside the LA (e.g., to the LAA) depends on where the transseptal puncture is formed on the fossa ovalis. In the disclosed technique, a processor detects a location in the fossa ovalis that provides the most comfortable access to the LAA without requiring excessive bending of the catheter. This location may be determined based on imaging—4D intracardiac echography (ICE) or CT scanning—to identify the LAA location, as well as known bending properties of the transseptal catheter.

The processor obtains three positions that are registered with respect to each other to enable reaching the correct position in the fossa ovalis: 1. target location (e.g., at entrance to LAA), 2. septum candidate location over the fossa ovalis, and 3. location of the device relative to a trajectory that the catheter should take based on the bending properties (e.g., achievable radius of curvature) of the catheter.

The processor can mark the location of the device on the 4D ICE image, or otherwise guide the physician to the detected location based on 3D position coordinates from a magnetic sensor on the catheter and registration between the imaging device and the sensor. The sheath, an LAA landing site (e.g., a location over an entry surface), and the transseptal puncture point (also called “access”) can all be visualized in a 3D mapping system (e.g., CARTO®).

Another disclosed technique that assumes accessing the RA via the lower vena cava (LVC) utilizes a clinical strategy of directly aiming the catheter tip to the left superior pulmonary vein (LSPV) to ensure safe puncturing. The technique overcomes the challenge of safely introducing the sheath and catheters to the LA, e.g., without a perforation hazard from puncturing critical organs in the vicinity, such as the aorta, LA posterior wall and the esophagus.

Using the direct aiming approach, the catheter tip (e.g., a piercing device) is positioned and oriented inside the RA such that the LSPV ostium is in direct aim of the piercing device (e.g., needle) that is advanced toward the fossa ovalis. When the puncture position is deemed as correct, the catheter is advanced to puncture the septum therein.

The disclosed technique provides the physician with real-time optimal catheter position and orientation for the puncture by using an anatomical map, such as one produced using an US catheter (i.e., using 4D ICE) so a processor can both identify the anatomy and automatically plan the safest access path. Accuracy can be improved by integrating catheter position and orientation information with the US image using a magnetic position sensor on the catheter.

After performing the transseptal puncture, a further step may be inserting the 4D ICE probe into the LA in order to accurately map the clinical target (e.g., LAA) for use with a catheter guiding software. Such intra-LA mapping with the US catheter enables very accurate measurement of the anatomy (e.g., of the LAA), and enables a physician using the guiding software to select the most accurate treatment device (e.g., the best suited LAA occlusion device) in an informative way for the specific clinical scenario.

Typically, the processor is programmed in software containing a particular algorithm that enables the processor to conduct each of the processor-related steps and functions outlined above.

### System Description

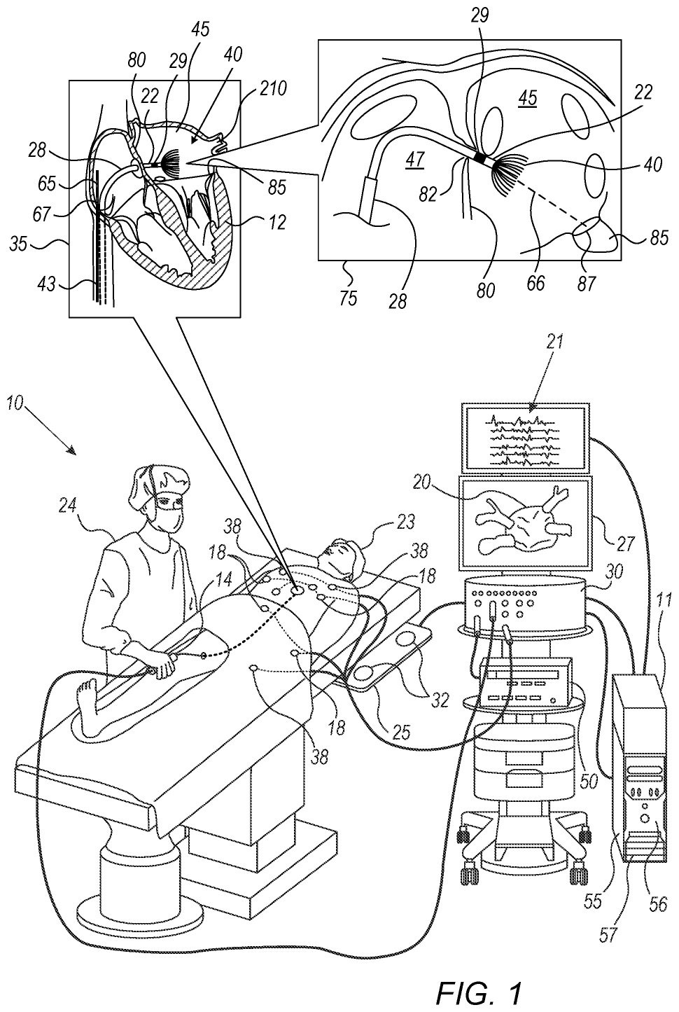

FIG. 1 is a schematic, pictorial illustration of a catheterization system 10 comprising a catheter 14 carrying a device 40 inside the left atrium (LA) 45, the catheter inserted into the LA using a transseptal access 82, in accordance with an example of the present disclosure. By way of example, device 40 is a left atrial appendage (LAA) occlusion device. Further seen is ultrasound probe 43 (e.g., an intracardiac ultrasound (US) catheter 43).

A distal end of a shaft 22 of catheter 14 is inserted by a physician 24 through a sheath 28 into a left atrium 45 of heart 12, seen in insets 35 and 75, of a patient 23 lying on a table. During the insertion of shaft 22, LAA occlusion device 40 is maintained in a collapsed configuration by sheath 28. By containing LAA occlusion device 40 in a collapsed configuration, sheath 28 also serves to minimize vascular trauma along the way to the target location.

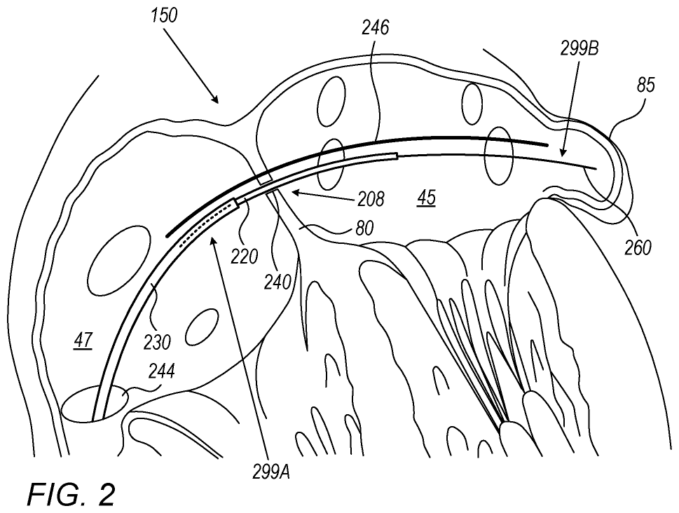

To reach LAA 85 inside LA 45, seen in insets 35 and 75, physician 24 first navigates sheath 28 to an inferior vena cava 244 access approach of a right atrium 47. The physician accesses LA 45 using a hole 82 (also called “transseptal access”) pierced with one of the two methods described in FIGS. 2-3 and 4-5, a septum 80 dividing the atria.

When inside LAA 85, the physician advances a distal end of a shaft 22 via sheath 28 and deploys LAA occlusion device 40, which is coupled to a distal edge of the shaft inside LAA 85.

As further seen in inset 75, to successfully access LAA 85, the physician aligns the sheath 28 at a particular direction 66 inside LA 45 that points to an ostium 87 of LAA 85.

To guide catheter 14 and to image anatomical landmarks inside heart 12, the physician uses ultrasound imaging catheter 43 (shown in an inset 35) comprising an ultrasound array 65 and a position sensor 67. Ultrasound array 65 can be 1D or 2D in order to generate 2D or 3D ultrasound images, respectively. Imaged target anatomical structures (e.g., septum 80 and LAA 85) are presented to physician 24 by a processor on a display device 27, e.g., as a volume rendering (also called hereinafter “anatomical map”). References to a “volume rendering” or “anatomical map,” as used herein, shall be understood to refer to a computational model stored in the memory of a computer processing system, whether or not actually displayed to a user, i.e., “rendered,” via a user interface, or merely utilized by the processor for computational purposes, such as identifying a targeted anatomical location or trajectory between anatomical locations.

Integral position sensor 67 is preregistered with array 65 of catheter 43. Specifically, sensor 67 is configured to output signals indicative of a position and orientation of the ultrasound transducer array 65 inside heart 12. A processor of the system is configured to use the sensor's signal output to acquire one or more ultrasound images of anatomical structures oriented in various respective orientations relative to ultrasound transducer array 65.

Magnetic-based position sensor 67 may be operated together with a location pad 25 that includes a plurality of magnetic coils 32 configured to generate magnetic fields in a predefined working volume. Real-time position of the distal ends of catheter 43, and of catheter 14, may be tracked based on magnetic fields generated with location pad 25 and sensed by magnetic-based position sensors 67 and 29, respectively. Details of the magnetic-based position sensing technology are described in U.S. Pat. Nos. 5,539,199; 5,443,489; 5,558,091; 6,172,499; 6,239,724; 6,332,089; 6,484,118; 6,618,612; 6,690,963; 6,788,967; 6,892,091.

System 10 includes one or more electrode patches 38 positioned for skin contact on patient 23 to establish a location reference for location pad 25 as well as impedance-based tracking of electrodes 26. For impedance-based tracking, electrical current is directed toward electrodes 26 and sensed at electrode skin patches 38 so that the location of each electrode can be triangulated via electrode patches 38. Details of the impedance-based location tracking technology are described in U.S. Pat. Nos. 7,536,218; 7,756,576; 7,848,787; 7,869,865; and 8,456,182.

A recorder 11 displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more electrodes at a distal tip of a third catheter (not shown) configured for ablation. The third catheter may be used (e.g., in an alternative procedure to the shown LAA 85 occlusion) to ablate an ostium of a pulmonary vein (PV), such as of the left superior PV (LSPV) 210, to eliminate an arrhythmia. Energy produced by ablation energy generator 50 may include, but is not limited to, radiofrequency (RF) energy or pulsed-field ablation (PFA) energy, including monopolar or bipolar high-voltage DC pulses to effect irreversible electroporation (IRE), or combinations thereof.

Patient interface unit (PIU) 30 is an interface configured to establish electrical communication between catheters, electrophysiological equipment, power supply and a workstation 55 for controlling the operation of system 10. Electrophysiological equipment of system 10 may include, for example, multiple catheters, location pad 25, body surface ECG electrodes 18, electrode patches 38, ablation energy generator 50, and recorder 11. Optionally, and preferably, PIU 30 additionally includes processing capability for implementing real-time location computation of the catheters and for performing ECG calculations.

Workstation 55 includes memory 57, processor unit 56 with memory or storage with appropriate operating software loaded therein, and user interface capability. Workstation 55 may provide multiple functions, optionally including (1) modeling endocardial anatomy in three-dimensions (3D) and rendering the model or an anatomical map 20 for display on display device 27, (2) displaying activation sequences (or other data) compiled from recorded electrograms 21 in representative visual indicia or imagery included in the rendered anatomical map 20 on display device 27, (3) displaying real-time position and orientation of multiple catheters within the heart chamber, and (4) displaying sites of interest, such as places where ablation energy has been applied, on display device 27. One commercial product embodying elements of system 10 is available as the CARTO™ 3 System, available from Biosense Webster, Inc.

### Optimal Transseptal Puncture Location Using Curved Trajectory

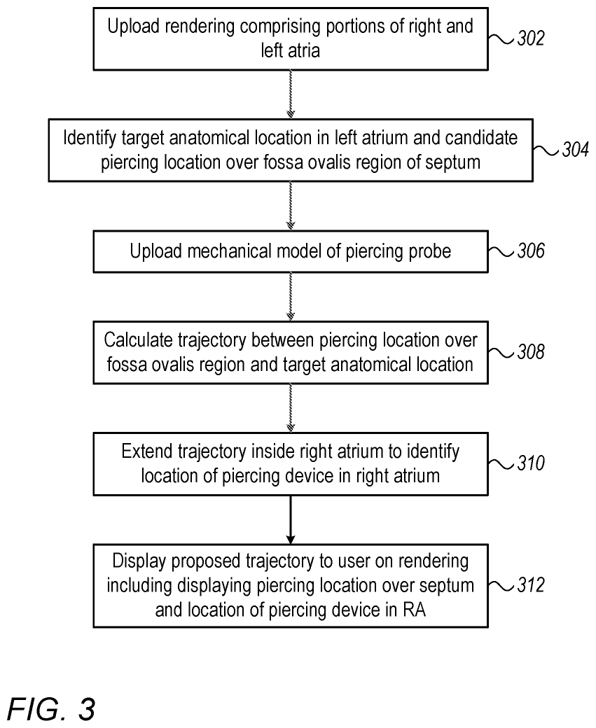

FIG. 2 is a schematic, pictorial illustration of a catheter 150 at an optimized transseptal puncture location 240, in accordance with an example of the present disclosure. In this example, puncture location 240 is an optimal location that a processor finds over the fossa ovalis region 208 of septum 80 by running the steps described below and in FIG. 3. The objective of the piercing procedure is to allow correct access point 240 to a treating catheter, such as catheter 14, to LAA 85.

FIG. 2 shows the end of the puncturing procedure, after location 240 is found and after the physician brings catheter 150 to a correct origin position 299A, and after the physician punctured septum 80 at location 240 by advancing a piercing guidewire 260 of catheter 150 via a sheath 230.

As seen, the trajectory 246 of catheter 150 is curved between the location 299A piercing guidewire 260 inside sheath 230 and its target location 299B inside LAA 85.

To find the optimal curve which passes via location 240, the disclosed technique requires the processor to obtain three positions: 1. target location 299B (e.g., at entrance to LAA), 2. septum location 240 over a trajectory that the catheter should take based on the bending properties (e.g., achievable radius of curvature) of the catheter, and 3. current location 299A of the device.

In practice, the physician advances a shaft 220 of catheter 150 with the piercing needle fitted at its distal end.

In some examples, the processor obtains location 299B in real time using an anatomical map, such as produced using the aforementioned US catheter 43 (i.e., using 4D ICE). This further obtains, from the US imaging, a current location of the distal end of sheath 230. Using the known properties of the catheter (sheath and shaft), the processor can overlay a candidate trajectory that must end in location 299B. The processor runs an algorithm that calculates location 299A and an orientation of the sheath at location 299A, so that, based on its mechanical properties, the trajectory ends at location 299B. The crossing point of this trajectory and fossa ovalis 208 region of septum 80 is optimal puncture location 240. The processor may tag locations 299A and 240 over the anatomical map, so that the physician can manipulate catheter 150 to achieve the proposed trajectory by manipulating the catheter and receiving real-time visual feedback from the US imaging.

The example of FIG. 2 is one of several possible. As another option, the system may be using a position-sensor navigated sheath to perform the procedure, instead or in addition to a sensor-based needle. In this case, the trajectory and location can be calculated based on the navigated sheath data. In other example the catheter includes a force sensor on the needle and the processor uses bending properties of the catheter and the force value to calculate the trajectory.

FIG. 3 is a flow chart that schematically describes a method of finding the optimized transseptal puncture location described in FIG. 2, in accordance with an example of the present disclosure. The algorithm, according to the presented example, carries out a process that begins with processor 56 uploading a rendering of at least part of a septum, including adjacent RA volume, and of an LA of a heart of a patient, at a rendering uploading step 302.

Next, processor 56 is used to identify a target anatomical location (e.g., at entrance 87 to LAA 85), and candidate piercing location 240 over fossa ovalis region 208 of septum 80, at an identification step 304.

At a mechanical model uploading step, the processor uploads a mechanical model of the piercing probe (being, for example, a catheter with a guidewire, or with a shaft fitted with a needle, either of which is advanced using a sheath of the catheter), at a mechanical model uploading step 306. Examples of the mechanical properties provided by the model include bending properties, such as a minimal radius of curvature and a maximal deflection angle of the transseptal catheter as a whole, or such mechanical properties achievable with each of its separate elements (e.g., the sheath, and the guidewire or shaft). The model may account for multiple stages of catheter deployment, such as when the shaft or guidewire is inside the sheath or is partially extended.

At a trajectory calculation step 308, processor 56 is used to calculate a trajectory inside the LA between candidate piercing location 240 and target anatomical location. This trajectory is typically curved.

Next, processor 56 is used to calculate an extension of the trajectory inside a right atrium, with which the probe is to be aligned prior to entering the left atrium, so as to identify a required location for the piercing device in the RA, at device location finding step 310.

Finally, at a displaying step 312, processor 56 displays the proposed trajectory (e.g., trajectory 246) on the rendering, including displaying the piercing location over the septum (e.g., location 240) and location of the piercing device in the RA (e.g., location 299A).

The example flow chart shown in FIG. 3 is chosen purely for the sake of conceptual clarity. In some examples, the above process is performed in real time during a clinical procedure. In such a case, the processor can identify a current location of the piercing device inside the RA (e.g., a guidewire 260 when still inside a distal end of sheath 220). The processor can mark the actual current location of the device on a 4D ICE image or otherwise guide the physician based on 3D position coordinates from a magnetic sensor on the piercing catheter (e.g., on the shaft and or the sheath) and registration between the imaging device and the magnetic sensor.

### Safe Transseptal Puncture Using Straight Line Trajectory

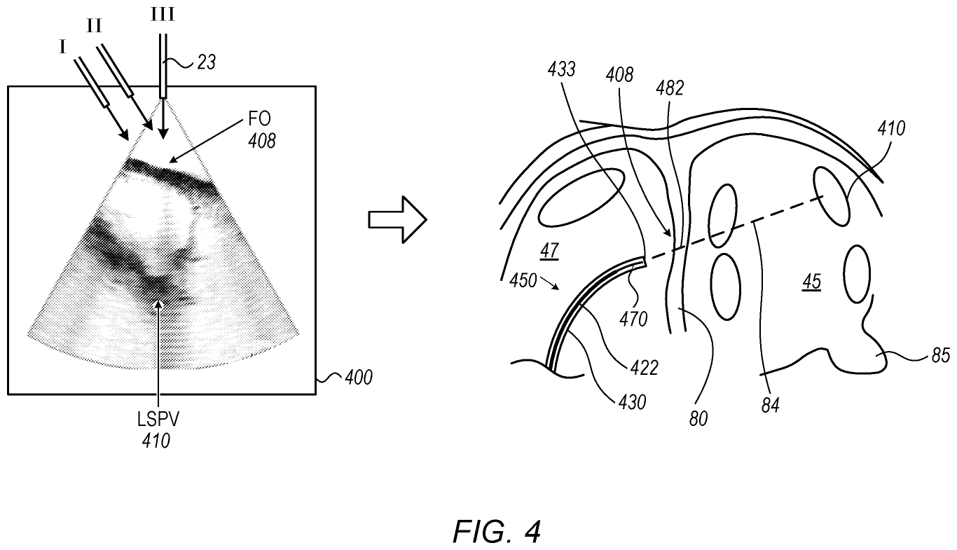

FIG. 4 is a schematic, pictorial illustration of a catheter 450 at an optimized transseptal puncture location 482, in accordance with another example of the present disclosure. The puncture is made in a location over the fossa ovalis region (shown in as a depression 408 in septum 80).

Because the puncture site is in the vicinity of the aorta, the heart posterior wall, and the esophagus, an incorrect approach may cause perforation. To mitigate risk, the disclosed technique guides the physician how to place and orient needle 470 inside RA 47, so that, once advanced from distal edge 433 of sheath 430 (using shaft 422), it assumes the chosen straight trajectory 84 between the piercing location 482 to the left superior pulmonary vein (LSPV) 410.

In the disclosed technique, the physician must aim the piercing needle 470 to the ostium of LSPV 410. To achieve this, processor 56 calculates the optimal position and orientation of needle 470 for the puncture. The process is done in real time, and the physician or a processor uses the aforementioned 4D ICE, such as real-time US image sequence 400, for guidance.

Using real-time US image 400, the physician can identify the anatomy (or the processor can automatically identify the anatomy) and plan the safest access path (in case of a processor, automatically plan the safest access path). As seen, between US images capturing steps I, II and III, the catheter is guided into a proper position and orientation inside RA 47. From probe position and orientation, as shown in step III, the physician can advance the needle in a line trajectory to LSPV 410 via piercing location 482 in fossa ovalis (FO) 408. Continuous real-time feedback ensures that the physician is well informed about the current probe position and orientation and how far are these from optimal.

The ICE allows a physician or a processor to navigate devices that lack position-tracking sensing by identifying and segmenting them in the 4D volume acquired in an ICE.

Using position-tracking sensors can improve accuracy of position and orientation guidance. To this end, spatial information on the probe obtained using a magnetic position and orientation sensor, such as sensor 29, can be integrated with the US image.

It was found by the inventors that the resulting piercing location 482 also provides useful access to a treatment catheter to treatment locations such as LAA 85.

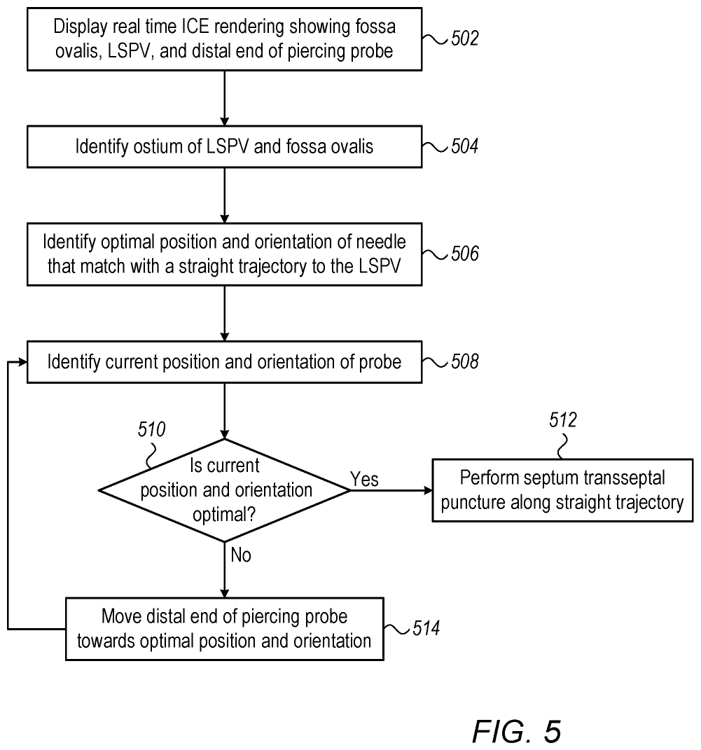

FIG. 5 is a flow chart that schematically describes a method of finding the optimized transseptal puncture location described in FIG. 4, in accordance with an example of the present disclosure. The algorithm, according to the presented example, carries out a process that begins at an ICE displaying step 502, with processor 56 displaying a real-time rendering (e.g., 4D ICE) of a fossa ovalis region of a septum, including adjacent RA volume, of an LA of a heart of a patient, including the ostium of the LSPV, and of the distal end of a piercing probe. An example of such a rendering is rendering 400.

Next, processor 56 is used to identify, on the rendering, the target anatomical location that is the LSPV, and the fossa ovalis region of septum, at an anatomy identification step 504.

At piercing probe optimal position and orientation identification step 506, the processor identifies an optimal position and orientation of a needle of the probe. One way to do this is to extend a linear trajectory between a center of the ostium of the LSPV and a center of the fossa ovalis region. The centers are defined, up to a given tolerance, and thus there is a tolerance over the line in space that connects the two anatomical landmarks. This tolerance is translated into a tolerance for the optimal position and orientation of the probe.

In one example, such a tolerance is displayed (e.g., overlayed on the US image) as a narrow cone of acceptance into which the physician has to bring the distal end of the probe.

Next, the physician, or a processor, identifies the actual (i.e., current) position and orientation of the piercing probe (e.g., of needle 470), at actual position and orientation identification step 508.

In a next checking step 510, the current position and orientation is compared (e.g., visually, or by a processor) to the optimal ones, up to a tolerance.

If the current position and orientation are optimal within tolerance, e.g., they are like those shown in step III on rendering 400, the physician may perform the septum transseptal puncture by advancing needle 470 in a straight trajectory toward the ostium of the LSPV, at a puncturing step 512.

If current position and orientation are not optimal within tolerance, e.g., they are like those shown in steps I or II on rendering 400, the physician or the processor moves the distal end of the piercing probe in order to improve position and orientation, as shown in steps I-III of rendering 400, and the process returns to step 508.

### EXAMPLES

### Example 1

A system (10) includes a display (27) and a processor (55). The display is configured to present a rendering (400) of at least part of a septum (80) and a left atrium (45) of a heart (12) of a patient. The processor is configured to (a) identify in the rendering the septum and a target anatomical location (299B, 410) to be reached by a probe (14, 150, 450) via the septum, (b) calculate a trajectory (84, 246) for the probe between the septum and the target anatomical location, including identifying over the septum an entrance location (82, 482) for the probe to cross the septum and reach the target anatomical location, and (c) present the entrance location to a user for penetrating the septum therein.

### Example 2

The system (10) according to example 1, wherein the probe (14, 150, 450) comprises a flexible catheter, and wherein the processor (55) is configured to calculate the trajectory based on specified bending properties of the catheter.

### Example 3

The system (10) according to any of examples 1 and 2, wherein the probe (14, 150, 450) comprises a transseptal needle (470), and wherein the trajectory (84, 246) is linear (84).

### Example 4

The system (10) according to any of examples 1 and 2, wherein the probe (14, 150, 450) comprises a transseptal guidewire (260).

### Example 5

The system (10) according to any of examples 1 through 3, wherein the processor (55) is further configured to calculate, and display on the rendering (400), an extension of the trajectory inside a right atrium that the probe is to be aligned with prior to entering the left atrium.

### Example 6

The system (10) according to any of examples 1 through 4, wherein the processor (55) is further configured to identify a target position and orientation of a piercing device (260, 470) coupled at a distal end of the probe along the trajectory.

### Example 7

The system (10) according to any of examples 1 through 6, wherein the rendering (400) is an ultrasound image (400) obtained using an invasive ultrasound probe (43).

### Example 8

The system (10) according to any of examples 1 through 7, wherein the processor (55) is configured to display the probe (14, 150, 450) on the ultrasound image (400).

### Example 9

The system (10) according to any of examples 1 through 8, wherein the processor (55) is further configured to identify a position and orientation of a distal end of the probe by using a sensor (29) fitted on the distal end of the probe.

### Example 10

The system (10) according to any of examples 1 through 9, wherein the target anatomical location is a left atrial appendage (LAA) (85) of the heart, and wherein the probe (14) is fitted with an LAA occlusion device (40).

### Example 11

The system (10) according to any of examples 1 through 9, wherein the target anatomical location is an ostium (410) of a superior left pulmonary vein (LSPV).

### Example 12

The system (10) according to any of examples 1 through 11, wherein the processor (55) is further configured to register with one another, and display over the rendering, (i) a current position of the probe, (ii) the entrance location and (iii) a target location at the target anatomical structure.

### Example 13

A method includes identifying in a rendering (400) (i) at least part of a septum (80) and a left atrium (45) of a heart (12) of a patient. The septum and a target anatomical location (299B, 410) are identified in the rendering, to be reached by a probe (14, 150, 450) via the septum. A trajectory is calculated for the probe between the septum and the target anatomical location, including identifying over the septum an entrance location (82, 482) for the probe to cross the septum and reach the target anatomical location. The entrance location is presented to a user for penetrating the septum therein.

It will thus be appreciated that the examples described above are cited by way of example, and that the present disclosure is not limited to what has been particularly shown and described herein above. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
a display configured to present a rendering of at least part of a septum and a left atrium of a heart of a patient; and
a processor, which is configured to:
identify in the rendering (i) the septum and (ii) a target anatomical location to be reached by a probe via the septum;
calculate a trajectory for the probe between the septum and the target anatomical location, including identifying over the septum an entrance location for the probe to cross the septum and reach the target anatomical location; and
present the entrance location to a user for penetrating the septum therein.

2. The system according to claim 1, wherein the probe comprises a flexible catheter, and wherein the processor is configured to calculate the trajectory based on specified bending properties of the catheter.

3. The system according to claim 1, wherein the probe comprises a transseptal needle, and wherein the trajectory is linear.

4. The system according to claim 1, wherein the probe comprises a transseptal guidewire.

5. The system according to claim 1, wherein the processor is further configured to calculate, and display on the rendering, an extension of the trajectory inside a right atrium that the probe is to be aligned with prior to entering the left atrium.

6. The system according to claim 1, wherein the processor is further configured to identify a target position and orientation of a piercing device coupled at a distal end of the probe along the trajectory.

7. The system according to claim 1, wherein the rendering is an ultrasound image obtained using an invasive ultrasound probe.

8. The system according to claim 7, wherein the processor is configured to display the probe on the ultrasound image.

9. The system according to claim 1, wherein the processor is further configured to identify a position and orientation of a distal end of the probe by using a sensor fitted on the distal end of the probe.

10. The system according to claim 1, wherein the target anatomical location is a left atrial appendage (LAA) of the heart, and wherein the probe is fitted with an LAA occlusion device.

11. The system according to claim 1, wherein the target anatomical location is an ostium of a superior left pulmonary vein (LSPV).

12. The system according to claim 1, wherein the processor is further configured to register with one another, and display over the rendering, (i) a current position of the probe, (ii) the entrance location and (iii) a target location at the target anatomical structure.

13. A method, comprising:
identifying in a rendering (i) a septum and (ii) a target anatomical location to be reached by a probe via the septum;
calculating a trajectory for the probe between the septum and the target anatomical location, including identifying over the septum an entrance location for the probe to cross the septum and reach the target anatomical location; and
presenting the entrance location to a user for penetrating the septum therein.

14. The method according to claim 13, wherein the probe comprises a flexible catheter, and wherein the processor is configured to calculate the trajectory based on specified bending properties of the catheter.

15. The method according to claim 13, wherein the probe comprises a transseptal needle, and wherein the trajectory is linear.

16. The method according to claim 13, wherein the probe comprises a transseptal guidewire.

17. The method according to claim 13, and comprising calculating, and displaying on the rendering, an extension of the trajectory inside a right atrium that the probe is to be aligned with prior to entering the left atrium.

18. The method according to claim 13, and comprising identifying a target position and orientation of a piercing device coupled at a distal end of the probe along the trajectory.

19. The method according to claim 13, wherein the rendering is an ultrasound image obtained using an invasive ultrasound probe.

20. The method according to claim 19, and comprising displaying the probe on the ultrasound image.

21. The method according to claim 13, wherein identifying a position and orientation of a distal end of the probe comprises using a sensor fitted on the distal end of the probe.

22. The method according to claim 13, wherein the target anatomical location is a left atrial appendage (LAA) of the heart, and wherein the probe is fitted with an LAA occlusion device.

23. The method according to claim 13, wherein the target anatomical location is an ostium of a superior left pulmonary vein (LSPV).

24. The method according to claim 13, and comprising registering with one another, and display over the rendering, (i) a current position of the probe, (ii) the entrance location and (iii) a target location at the target anatomical structure.
